(19) United States
(12) Patent Application Publication
(10) Pub. No.: US 2003/0191131 A1
(43) Pub. Date: Oct. 9, 2003

(54) Use of organic compounds

(76) Inventor: Michael Heinrich, Lake Oswego, OR (US)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/395,525

(22) Filed: Mar. 24, 2003

Related U.S. Application Data
(60) Provisional application No. 60/370,331, filed on Apr. 5, 2002.

(51) Int. Cl. A61K 31/506

(52) U.S. Cl. 514/252.18

(57) ABSTRACT

4-(4-methylpiperazin-1-ylmethyl)-N-[4-methyl-3-(4-pyridin-3-yl)pyrimidin-2-ylamino)phenyl]-benzamide of the formula I

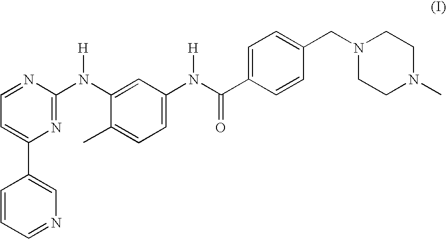

or a pharmaceutically acceptable salt thereof can be used in the treatment of canine mast cell neoplasms.

The present invention pertains to the field of veterinary medicine, and in particular to veterinary oncology.

The invention more specifically relates to the use of 4-(4-methylpiperazin-1-ylmethyl)-N-[4-methyl-3-(4-pyridin-3-yl)pyrimidin-2-ylamino)phenyl]-benzamide (hereinafter “COMPOUND I”) or a pharmaceutically acceptable salt thereof for the manufacture of pharmaceutical compositions for use in the treatment of canine mast cell neoplasms, to the use of COMPOUND I or a pharmaceutically acceptable salt thereof in the treatment of canine mast cell neoplasms, and to a method of treating warm-blooded animals including dogs suffering from canine mast cell neoplasms by administering to a said animal in need of such treatment an effective dose of COMPOUND I or a pharmaceutically acceptable salt thereof.

Mast cell neoplasms occur in both humans and animals. In dogs, mast cell neoplasms are called mastocytomas, and the disease is common, representing 7-21% of canine tumors. A distinction must be drawn between human mastocytosis, which is usually transient or indolent, and canine mast cell neoplasia, which behaves unpredictably and is often aggressive and metastatic. For instance, human solitary mastocytomas essentially never metastasize; in contrast, ≈50% of canine mastocytomas behave in a malignant fashion, as estimated by Hottendorf & Nielsen (1969) after review of 46 published reports of tumors in 938 dogs.

Cancer in the pet population is a spontaneous disease. Pet owners, motivated by prolonging the quality of their animals' life, frequently seek out the specialized care and treatment of veterinary oncologists at private referral veterinary hospitals and veterinary teaching hospitals across the country. Therapeutic modalities of veterinary cancer patients are similar to humans, including surgery, chemotherapy, radiation therapy and biotherapy. It has been estimated that there are 42 million dogs and approximately 20 million cats in the United States. Using crude estimates of cancer incidence, there are roughly 4 million new cancer diagnoses made in dogs and a similar number in cats made each year.

Cutaneous mast cell tumors in dogs are a common problem. Most mast cell tumors are benign and are cured with simple resection; however, if recurrent or metastatic to distant sites therapeutic options are limited. Treatment options for recurrent lesions can include external beam radiation therapy. For distant metastases or disseminated disease the use of Lomustine® and vinblastine containing chemotherapy protocols have demonstrated some benefit. Sites for metastases for mast cell tumors include skin, regional lymph nodes, spleen, liver and bone marrow.

The KIT receptor's involvement in the pathogenesis of mastocytosis is suggested by the observation that several mutations resulting in constitutive activation of KIT have been detected in a number of mast cell lines. For instance, a point mutation in human c-KIT, causing substitution of Val for Asp816 in the phosphotransferase domain and receptor autoactivation, occurs in a long-term human mast cell leukemia line (HMC-1) and in the corresponding codon in two rodent mast cell lines. Moreover, this activating mutation has been identified in situ in some cases of human mastocytosis. Two other activating mutations have been found in the intracellular juxtamembrane region of KIT, i.e., the Val560Gly substitution in the human HMC-1 mast cell line, and a seven amino acid deletion (Thr573-His579) in a rodent mast cell line called FMA3.

It has now surprisingly been demonstrated that canine mast cell neoplasms can be successfully treated with COMPOUND I or pharmaceutically acceptable salt thereof.

COMPOUND I is 4-(4-methylpiperazin-1-ylmethyl)-N-[4-methyl-3-(4-pyridin-3-yl)pyrimidin-2-ylamino)phenyl]-benzamide having the formula I

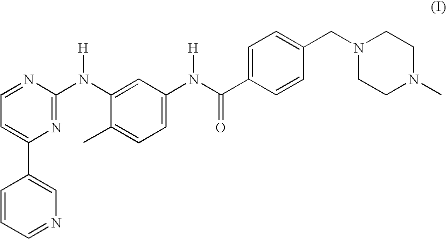

The preparation of COMPOUND I and the use thereof, especially as an anti-tumour agent, are described in Example 21 of European patent application EP-A-0 564 409, which was published on Oct. 6, 1993, and in equivalent applications and patents in numerous other countries, e.g., in U.S. Pat. No. 5,521,184 and in Japanese patent 2706682.

Pharmaceutically acceptable salts of COMPOUND I are pharmaceutically acceptable acid addition salts, like for example with inorganic acids, such as hydrochloric acid, sulfuric acid or a phosphoric acid, or with suitable organic carboxylic or sulfonic acids, for example, aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic acid, or aromatic sulfonic acids, for example benzene-, p-toluene- or naphthalene-2-sulfonic acid.

The monomethanesulfonic acid addition salt of COMPOUND I (hereinafter “SALT I”) and a preferred crystal form thereof are described in PCT patent application WO99/03854 published on Jan. 28, 1999.

Depending on species, age, individual condition, mode of administration and the clinical picture in question, effective doses, for example, daily doses of about 20-200 mg, preferably 80-160 mg, especially 125 mg, are administered to warm-blooded animals of about 5 kg bodyweight. For adult dogs of about 5 kg with unresectable and/or metastatic malignant canine mast cell neoplasms, a starting dose of 125 mg daily can be recommended. For dogs with an inadequate response after an assessment of response to therapy with 125 mg daily, dose escalation can be safely considered and dogs may be treated as long as they benefit from treatment and in the absence of limiting toxicities. Dosages may be titered so as to achieve plasma levels of at least 0.2 μM (micromolar), preferably at least 0.5 μM, more preferably at least 1 μM. Achieving and/or maintaining a plasma level of about 1 μM is particularly preferred.

The invention relates also to a method for administering to a dog subject having canine mast cell neoplasms COMPOUND I or a pharmaceutically acceptable salt thereof, which comprises administering a pharmaceutically effective amount of COMPOUND I or a pharmaceutically acceptable salt thereof to the dog once daily for preferably a period exceeding 1 month, 2 months or even 3 months. The invention relates especially to such method wherein a daily dose of about 20-200 mg, preferably 80-160 mg, especially 125 mg of SALT I is administered.

The invention will now be described with respect to the following examples; however, the scope of the present invention is not to be limited thereby.

### EXAMPLE 1

### Methods

Reagents: Novartis Pharma; Basel, Switzerland provided SALT I for use in these experiments. Fresh 10 mM stock solutions of the inhibitor were made before each experiment by dissolving compound in 1 mL Phosphate-Buffered Saline (PBS; Gibco-BRL).

Antibodies: A polyclonal rabbit anti-KIT antibody (c-kit Ab-1) was used at a dilution of 1:500 (c-kit Ab-1; Oncogene, Cambridge, Mass.). An anti-phosphotyrosine antibody (PY20) was used at a dilution of 1:1000 (PY20 Transduction Laboratories; Lexington, Ky.). Peroxidase conjugated goat anti-mouse antibody was used at a dilution of 1:5000 and goat anti-rabbit antibody at a dilution of 1:10,000 (Pierce; Rockford, Ill.).

Cell lines: BR and C2 canine mastocytoma cells lines were obtained from Dr. George Caughey (University of California at San Francisco, San Francisco, Calif.). Both cell lines were maintained in DMEM supplemented with 2% bovine calf, 1 mM L-glutamine, 12.5 mM HEPES (pH 7.5), 0.25 mg/mL Histidine, 1% Penicillin-Streptomycin and 1% fungizone. The BR and C2 cells were derived from canine mast cell tumors and were originally established in long-term culture after initial passaging in immunodeficient mice (DeVinney et al., Am. J. Respir. Cell Mol. Biol., Vol. 3, No. 5, pp. 413-420 (1990); Lazarus et al., Am. J. Physiol., Vol. 251, No. 6, Pt 1, pp. C935-C944 (1986)). The BR cell line has a point mutation (T1752C) resulting in a Leucine to Proline substitution at amino acid 575 (juxtamembrane domain). The C2 cell line has an internal tandem duplication (ITD) of the KIT juxtamembrane region. The translation of this ITD results in reduplication of amino acid residues at the 3′ end of exon 11 (London et al., Exp. Hematol., Vol. 27, No. 4, pp. 689-697 (1999); Ma et al., J. Invest. Dermatol., Vol. 112, No. 2, pp. 165-170 (1999)).

Proliferation assays: Cells were added to 96-well plates at a density of 40,000 cells/well in normal culture media and varying concentrations of SALT I. Proliferation was measured at 48-72 hours using an XTT-based assay (Roche Molecular Biochemicals; Indianapolis, Ind.) (Heinrich et al., Blood, Vol. 96, No. 3, pp. 925-932 (2000).

Protein lysates: BR and C2 cells were washed ×2 in PBS and then quiesced in Optimem (Gibco-BRL) at 37° C. for approximately 18 hours. Cells were then incubated for 90 minutes in the presence of various concentrations of SALT I. Following this incubation, the cells were pelleted and lysed using 100-250 μL of protein lysis buffer (50 mM Tris, 150 mM NaCl, 1% NP-40, 0.25% Deoxycholate, with addition of the inhibitors aprotinin, leupeptin, pepstatin, PMSF and sodium orthovanadate [Sigma]). Western immunoblot analysis was performed as previously described (Hoatlin et al., Blood, Vol. 91, No. 4, pp. 1418-1425 (1998); Heinrich et al., Blood, Vol. 96, No. 3, pp. 925-932 (2000).

### EXAMPLE 2

### COMPOUND I Inhibits the Constitutively Activated KIT Kinase Associated with Canine Mast Cell Tumors

To test the efficacy of COMPOUND I in inhibiting the kinase activity of mutant forms of canine KIT we used two cells lines (BR and C2) that express two different constitutively activated KIT isoforms. The KIT mutations in these cell lines are both located in the juxtamembrane domain and are homologous to mutations seen in human Gastrointestinal Stromal Tumors (GISTs) (Lux et al., Am. J. Pathol., Vol. 156, No. 3, pp. 791-795 (2000); Rubin et al., Cancer Res., Vol. 61, No. 22, pp. 8118-8121 (2001). Lysates prepared from BR or C2 cells were probed with an anti-P-Tyr antibody and KIT receptor activation was assessed by measuring autophosphorylation. As reported previously, KIT autophosphorylation in these cells was observed in the absence of SLF (Ma et al., J. Invest. Dermatol., Vol. 112, No. 2, pp.165-170 (1999); Ma et al., J. Invest. Dermatol., Vol.114, No. 2, pp. 392-394 (2000)). Inhibition of KIT autophosphorylation by COMPOUND I was dose dependent with complete inhibition observed using 10 and 1.0 μM doses. Near complete inhibition was seen using a dose of 0.1 μM. Limited autophosphorylation of c-kit was seen using 0.001-0.01 μM doses of COMPOUND I. Thus, COMPOUND I not only inhibits the autophosphorylation of the mutated c-kit receptor in these cells, but also is a more potent inhibitor of this mutated receptor than it is of the wild type c-kit receptor (IC₅₀ 100-200 nM) (Heinrich et al., Blood, Vol. 96, No. 3, pp. 925-932 (2000)). To determine if COMPOUND I modulated expression of KIT protein, the membrane was stripped and reprobed with an anti-c-kit antibody. There was no change in expression of c-kit protein in of COMPOUND I treated cells. Therefore, COMPOUND I decreases autophosphorylation of mutant canine KIT polypeptide by inhibiting KIT kinase activity rather than by down regulating expression of KIT protein.

### EXAMPLE 3

### COMPOUND I Inhibits the Proliferation of Cell Lines of Canine Mast Cell Tumors

To test the biologic effect of inhibiting the kinase activity of a mutant c-kit receptor, we cultured BR or C2 cells for 48-72 hours in the presence of various concentrations of COMPOUND I. At inhibitor concentrations of 0.1-10 μM, proliferation was decreased by 90-95% compared to cells treated with media only. Partial inhibition of proliferation was seen at doses of 0.001-0.01 μM COMPOUND I. The decrease in proliferation seen with doses of 0.01-10 μM inhibitor was statistically significant (p<0.001). Therefore, COMPOUND I inhibits proliferation of BR and C2 cells with the same dose response range as seen for inhibition of receptor autophosphorylation. Morphologic observations of the inhibitor treated cells revealed changes consistent with apoptosis (data not shown).

TABLE 1
BR cells

| | Averages | % | SD | % SD |
|---|---|---|---|---|
| Cells | 0.929 | 100% | 0.030447 | 3% |
| 5 μM | 0.083 | 9% | 0.001732 | 0% |
| 1 μM | 0.105 | 11% | 0.002 | 0% |
| .1 μM | 0.105 | 11% | 0.002082 | 0% |
| .01 μM | 0.479 | 52% | 0.043016 | 5% |
| .001 μM | 0.781 | 84% | 0.033081 | 4% |

TABLE 1
C2 cells

| | Averages | % | SD | % SD |
|---|---|---|---|---|
| Cells | 1.236 | 100% | 0.04417 | 4% |
| 5 μM | 0.032 | 3% | 0.005686 | 0% |
| 1 μM | 0.037 | 3% | 0.013868 | 1% |
| .1 μM | 0.028 | 2% | 0.003512 | 0% |
| .01 μM | 0.754 | 61% | 0.185236 | 15% |
| .001 μM | 1.065 | 86% | 0.055245 | 4% |

Tables 1 and 2: BR or C2 cells were plated in 96-well plates at a concentration of 40,000 cells/well and cultured in normal growth media and varying concentration of COMPOUND I. Cellular proliferation was measured at 72 hours using an XTT-based assay system. Each COMPOUND I concentration was assayed in triplicate. Results are expressed as a percent of maximal proliferation (cells only, no COMPOUND I)±1 standard deviation. Representative results from one of six independent experiments are shown.

### EXAMPLE 4

### Capsules with 4-[(4-methyl-1-piperazin-1-ylmethyl)-N-[4-methyl-3-[[4-(3-pyridinyl)-2-pyrimidinyl]amino]phenyl]benzamide methanesulfonate, β-Crystal Form

Capsules containing 11.95 mg of the compound named in the title (=SALT I) corresponding to 10.0 mg of COMPOUND I (free base) as active substance are prepared in the following composition:

Composition

| | | |
|---|---|---|
| SALT I | 11.95 | mg |
| Celiulose MK GR | 9.2 | mg |
| Crospovidone XL | 1.5 | mg |
| Aerosil 200 | 0.2 | mg |
| Magnesium stearate | 0.15 | mg |
| | 23.0 | mg |

The capsules are prepared by mixing the components and filling the mixture into hard gelatin capsules, size 1.

### EXAMPLE 5

### Capsules with 4-[(4-methyl-1-piperazin-1-ylmethyl)-N-[4-methyl-3-[[4-(3-pyridinyl)-2-pyrimidinyl]amino]phenyl]benzamide methanesulfonate, β-Crystal Form

Capsules containing 10.0 mg of the compound named in the title (=SALT I) as active substance are prepared in the following composition:

Composition

| | | |
|---|---|---|
| Active substance | 10.0 | mg |
| Avicel | 20.0 | mg |
| PVPPXL | 1.5 | mg |
| Aerosil | 0.2 | mg |
| Magnesium stearate | 0.15 | mg |
| | 31.85 | mg |

The capsules are prepared by mixing the components and filling the mixture into hard gelatin capsules, size 1.

### EXAMPLE 6

### Example of a Prospective Case Series of Pet Dogs with Measurable Cutaneous Mast Cell Tumors

The study patients are pet dogs with measurable and histologically-confirmed mast cell tumors. Cases are limited to those with measurable lesions amenable to biopsy.

Eligibility criteria are:

Histologically-confirmed measurable cutaneous mast cell tumors

Cases will require serial biopsy with 2 mM Keyes punch before and during therapy

Histological grade (II-intermediate or III-poorly differentiated)

Performance status 0 or 1 (Modified Karnofsky—Table 3)

Informed owner consent

Exclusion criteria are:

Concurrent cytotoxic chemotherapy

Prednisone and non-steroidal anti-inflammatory drugs may not be initiated within 30 days of the study; if prednisone or non-steroidal anti-inflammatory drugs have been administered for greater than 30 days they may be continued

Abnormal serum bile acid test (liver function)

TABLE 3
Performance Status (Modified Karnofsky)

| Grade | Description |
|---|---|
| 0 | Normal activity |
| 1 | Restricted activity; decreased activity from pre-disease status |
| 2 | Compromised; ambulatory only for vital activities; consistently |
| | defecates and urinates in acceptable areas |
| 3 | Disabled; must be force fed; unable to confine urination and |
| | defecation to acceptable areas |
| 4 | Dead |

Pretreatment evaluation of all cases include physical examination, complete blood count, buffy coat, serum biochemistry, urinalysis, serum bile acids (fasting and post-prandial), documentation of regional lymph node size, abdominal radiographs and abdominal ultrasound. The treatment regimen is 25 mg/kg PO QD×60 days of SALT I.

Treatment is continued in all cases for 60 days unless disease progression is noted. In cases experiencing partial response or complete response ongoing therapy for an additional 60 days may be considered. Cases successfully completing therapy are eligible for repeat entry to study.

TABLE 4
Treatment and Clinical Evaluation Plan

| Action | Day 0 | Day 7 | Day 14 | Day 28 | q14 days |
|---|---|---|---|---|---|
| Clinical staging¹ | X | | | X | X |
| Physical examination | X | X | X | X | X |
| Measurement of tumor burden² | X | X | X | X | X |
| Start SALT I 25 mg/kg QD | X | | | | |
| Pharmacokinetics³ | X | | | | |
| Incisional biopsy⁴ | X | | | X | |
| Repeat Staging | | | | X | |

¹Initial staging consisis of physical examination, CBC, buffy coat, serum biochemistry, liver function tests (serum bile acids), urinalysis, abdominal radiographs, and abdominal ultrasound. Re-evaluation of may consist of physical examination and measurement of tumor burden alone or repeat clinical staging.
²Tumor burden is measured at days 0, 7, 14 and 28, and then every 14 days. Treatment response will be defined against measurable cutaneous lesion(s) and other lesions identified at staging (CR, PR, SD, PD - defined below).
³Collection of plasma from the first 5 entered cases is undertaken at 0, 0.5, 1, 2, 5, 8, 12, 16 and 24 hours following first dose of SALT I.
⁴Incisional biopsy from defined measurable lesion(s) will be collected on days 0 and 28 from all cases. Additional biopsies are collected at the time of PR and after objective CR.

The efficacy of COMPOUND I is assessed against measurable cutaneous mast cell tumors, using clinical endpoints. Biological endpoints may be taken from serial biopsies collected from cutaneous tumors and from blood samples available through the treatment course.

Clinical endpoints include response rate of measurable tumors, objective response against measurable tumor, and time to progression of measurable tumor. All adverse side effects will be recorded.

“Objective Tumors Responses”, as defined below, are observed under treatment with COMPOUND I and indicate efficacy of the treatment regimen.

In particular, Complete Responses (CR) and Partial Responses (PRs) to treatment with COMPOUND I may be observed. Furthermore, it may be observed that more animals obtaining treatment show Stable Disease (SD), while less treated animals show Progressive Disease (PD). Also, it may be observed that less animals obtaining treatment show Relapse (R) of disease as compared to non-treated animals. Time To Progression (TTP), Duration of Remission and Survival may increase in animals under treatment with COMPOUND I.

“CR” is defined as disappearance of all clinical evidence of cancer and of any signs related to the cancer.

“PR” is defined as a 50% or greater decrease in the sum of the products of measurements for representative lesions, without an increase in size of any lesions or appearance of any new lesions.

“SD” is defined as no response or a response of less than that defined for PR or PD without appearance of any new lesions or worsening of clinical signs.

“PD” is defined as an unequivocal increase of at least 50% in the size of any measurable lesion or appearance of new lesions.

“R” is defined as appearance of new lesions or reappearance of old lesions in dogs that had had a CR; in dogs that had had only a PR, R was defined as at least a 50% increase in the sum of the products of measurements of representative lesions, compared with measurements obtained at the time of maximum response.

“TTP” is reported from day 0 of the protocol. TTP will be defined as the number of days start of therapy (from day 0) to R.

“Duration of Remission” is defined as the number of days from the objective response (PR or CR) to relapse.

“Survival” is defined as the number of days from the start of treatment with COMPOUND I to death. Cause of death will be noted but may include disease progression, toxicity and other.

1. The use of 4-(4-methylpiperazin-1-ylmethyl)-N-[4-methyl-3-(4-pyridin-3-yl)pyrimidin-2-ylamino)phenyl]-benzamide of the formula I 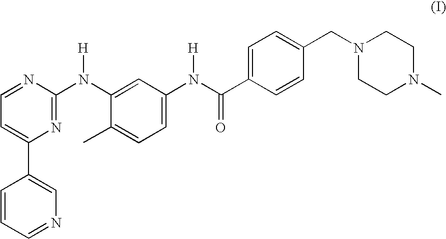
or a pharmaceutically acceptable salt thereof for the manufacture of pharmaceutical compositions for use in the treatment of canine mast cell neoplasms.

2. The use of 4-(4-methylpiperazin-1-ylmethyl)-N-[4-methyl-3-(4-pyridin-3-yl)pyrimidin-2-ylamino)phenyl]-benzamide of the formula I 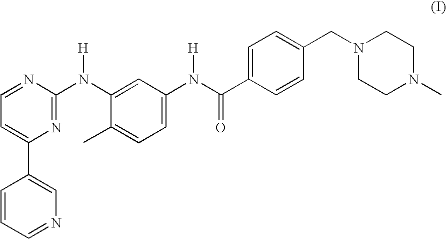
or a pharmaceutically acceptable salt thereof in the treatment of canine mast cell neoplasms.

3. A method of treating dogs suffering from canine mast cell neoplasms which comprises administering to a said dog in need of such treatment a dose, effective against canine mast cell neoplasms, of 4-(4-methylpiperazin-1-ylmethyl)-N-[4-methyl-3-(4-pyridin-3-yl)pyrimidin-2-ylamino)phenyl]-benzamide of the formula I 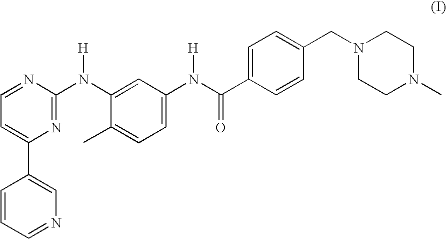
or a pharmaceutically acceptable salt thereof.

4. Use or method according to claim 3 wherein a pharmaceutically acceptable acid addition salt of 4-(4-methylpiperazin-1-ylmethyl)-N-[4-methyl-3-(4-pyridin-3-yl)pyrimidin-2-ylamino)phenyl]-benzamide of the formula I is administered.

5. Use or method according to claim 3 wherein a methanesulfonate salt of 4-(4-methylpiperazin-1-ylmethyl)-N-[4-methyl-3-(4-pyridin-3-yl)pyrimidin-2-ylamino)phenyl]-benzamide of the formula I is administered.

6. Use or method according to claim 3 wherein a daily dose of 20-200 mg of a monomethanesulfonate salt of 4-(4-methylpiperazin-1-ylmethyl)-N-[4-methyl-3-(4-pyridin-3-yl)pyrimidin-2-ylamino)phenyl]-benzamide of the formula I is administered to an adult dog.

7. Use or method according to claim 3 wherein a daily dose of a monomethanesulfonate salt of 4-(4-methylpiperazin-1-ylmethyl)-N-[4-methyl-3-(4-pyridin-3-yl)pyrimidin-2-ylamino)phenyl]-benzamide of the formula I is administered to a dog, and said daily dose comprises an amount of said monomethanesulfonate salt sufficient to maintain plasma levels of at least 0.2 μM.

8. A method for administering to a dog subject having canine mast cell neoplasms 4-(4-methylpiperazin-1-ylmethyl)-N-[4-methyl-3-(4-pyridin-3-yl)pyrimidin-2-ylamino)phenyl]-benzamide of the formula I 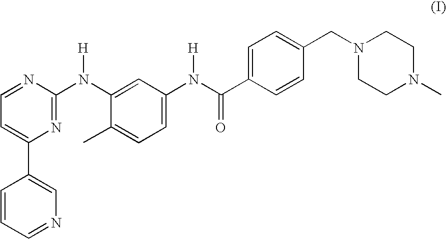
or a pharmaceutically acceptable salt thereof, which comprises administering a pharmaceutically effective amount of 4-(4-methylpiperazin-1-ylmethyl)-N-[4-methyl-3-(4-pyridin-3-yl)pyrimidin-2-ylamino)phenyl]-benzamide of the formula I or a pharmaceutically acceptable salt thereof to the dog subject once daily for a period exceeding 1 month.

9. A method according to claim 8 wherein a daily dose of 20-200 mg of the monomethanesulfonate salt of 4-(4-methylpiperazin-1-ylmethyl)-N-[4-methyl-3-(4-pyridin-3-yl)pyrimidin-2-ylamino)phenyl]-benzamide of the formula I is administered.
